(19) United States
(12) Patent Application Publication
(10) Pub. No.: US 2019/0328465 A1
(43) Pub. Date: Oct. 31, 2019

(54) SYSTEM AND METHOD FOR REAL IMAGE VIEW AND TRACKING GUIDED POSITIONING FOR A MOBILE RADIOLOGY OR MEDICAL DEVICE

(71) Applicants: AIH LLC, Victor, NY (US); Nordbo Robotics, Odense SO (DK)

(72) Inventors: Yan M. Li, Pittsford, NY (US); Jimmy Alison Jorgensen, Odense SO (DK); Chi Huang, Victor, NY (US)

(21) Appl. No.: 16/399,266

(22) Filed: Apr. 30, 2019

Related U.S. Application Data
(60) Provisional application No. 62/664,397, filed on Apr. 30, 2018.

(51) Int. Cl. A61B 34/20; A61B 6/12; G06T 7/00; A61B 6/00

(52) U.S. Cl. CPC A61B 34/20; A61B 6/12; G06T 7/0012; G16H 30/40; A61B 2034/2057; A61B 6/4441; A61B 6/547

(57) ABSTRACT

A location tracking system (LTS) together with a surgical area visualization (SAV) is responsible to guide a user such that multiple x-ray images can be taken from the same spot even though the x-ray machine is moved in between image captures. The LTS may be based on RGB-D data and the RGB-D sensor may be attached to the X-ray tube on the upper part of the arm looking downwards.

### CROSS-REFERENCE TO RELATED APPLICATIONS

This application claims the benefit of U.S. Provisional Patent Application No. 62/664,397, filed Apr. 30, 2018, entitled WEARABLE DEVICE FOR HEAD AND SPINE MONITORING AND CORRECTING, the entirety of which is incorporated herein by reference.

### FIELD OF THE INVENTION

The present invention relates to a location tracking system (LTS) together with a surgical area visualization (SAV) that are responsible for guiding a user such that multiple medical images, such as x-ray images, can be taken from the same spot even though the x-ray machine is moved in between image captures. The LTS may be based on RGB-D data and the RGB-D camera may be attached to the X-ray tube on the upper part of the arm looking downwards.

### BACKGROUND OF THE INVENTION

Medical devices, such as C-arm fluoroscopy, provide imaging information for guiding surgical procedures. Many orthopaedic, neurosurgical, vascular and trauma procedures typically require radiographic visualization of anatomy-specific views with surgical instrumentation and implants. In obtaining the desired view, repeated C-arm fluoroscopy images are often acquired, where radiology technicians use a trial-and-error approach of ‘fluoro hunting’ at the expense of time and radiation exposure to the patient as well as personnel. C-arm x-ray machines have been around for about 30 years but the design has not changed significantly in this regard.

Therefore, there is a need for a system and method with location tracking and surgical area visualization that will help better position the medical imaging device during medical practice like surgeries, reduce radiation and increase safety to patient and medical staffs, improve efficiency and accuracy and reduce the cost of operation.

### BRIEF SUMMARY OF THE INVENTION

The relative risk for cancer in the United States right now attributed to radiation-induced cancer is 2%-5%. Some medical staff even got cancer, like thyroid cancer or leukemia, at the end of their careers. The apparatus and method of the present invention aims for a 50%-80% reduction in radiation exposure to patients and medical staffs. Secondary, it will reduce time required for imaging and reduce overall surgery time and hospital cost. It is estimated to cost about $300/minute for an operative procedure, and is much more expensive for a patient under anesthesia (along with increased medical risks associated with a prolonged procedure). Currently, anywhere between about 5-25% of surgery time involves imaging depending the nature of procedure. The apparatus and method of the present invention reduces surgery time requirement by requiring fewer images before realignment of the imaging device is achieved.

It is, therefore, an aspect of the present invention to provide an intraoperative tracking and aligning apparatus for a medical imaging device comprising a depth camera configured to mount to the medical imaging device; a computing device including a processor and a memory, wherein the computing device is in communication with the depth camera; and a user interface in communication with the computing device. The depth camera acquires first image data and communicates the first image data to the computing device to display a first image on the user interface. The processor analyzes the first image data to identify a plurality of image features and superimposes a first alignment indicia over the first image. The depth camera then acquires second image data and communicates the second image data to the computing device. The processor analyzes the second image data to identify the plurality of image features and superimposes a second alignment indicia over the first image. One or both of a location or orientation of the medical imaging device is adjusted until the second alignment indicia coincides with the first alignment indicia.

In still another aspect of the present invention, a method for intraoperative tracking and aligning of a medical imaging device comprises a) mounting a depth camera to the medical imaging device; b) providing a computing device including a processor and a memory, wherein the computing device is in communication with the depth camera; c) providing a user interface in communication with the computing device; d) acquiring first image data using the depth camera; e) communicating the first image data to the computing device to display a first image on the user interface; f) analyzing the first image data, via the processor, to identify a plurality of image features; g) superimposing a first alignment indicia over the first image; h) acquiring second image data using the depth camera; i) communicating the second image data to the computing device; j) analyzing the second image data, via the processor, to identify the plurality of image features; k) superimposing a second alignment indicia over the first image; and l) adjusting one or both of a location or orientation of the medical imaging device until the second alignment indicia coincides with the first alignment indicia.

In another aspect of the system of the present invention, steps f) and j) further include transforming the plurality of image features within the first image data and the second image data, respectively, into camera coordinate space. Moreover, the first and second alignment indicia include first and second slider bars and a compass ball. The depth camera is configured to mount adjacent to an image intensifier of a C-arm x-ray machine with the image intensifier being located opposite an x-ray source. A camera angle of the depth camera relative to a travel direction of x-rays emitted by the x-ray source is adjustable, such as through the depth camera being mounted onto a swivel configured to allow adjustment of the camera angle. The computing device may also include the user interface.

Additional aspects, advantages and novel features of the present invention will be set forth in part in the description which follows, and will in part become apparent to those in the practice of the invention, when considered with the attached figures.

### BRIEF DESCRIPTION OF THE DRAWINGS

The accompanying drawings form a part of this specification and are to be read in conjunction therewith, wherein like reference numerals are employed to indicate like parts in the various views, and wherein:

FIG. 1 is a side plan view of an intraoperative tracking and aligning apparatus for a medical imaging device in accordance with an aspect of the present invention;

FIG. 2 is a front view of a camera suitable for use within the apparatus shown in FIG. 1;

FIG. 3 is a side plan view of the intraoperative tracking and aligning apparatus shown in FIG. 1 during use;

FIG. 4 is a plan view of a camera view showing camera coordinate space and first alignment indicia over a reference image;

FIG. 5 is a plan view of a camera view showing camera coordinate space and first and second alignment indicia over a reference image, wherein the indicia are poorly aligned; and

FIG. 6 is a plan view of a camera view showing camera coordinate space and first and second alignment indicia over a reference image, wherein the indicia are more closely aligned than in FIG. 5.

### DETAILED DESCRIPTION OF THE INVENTION

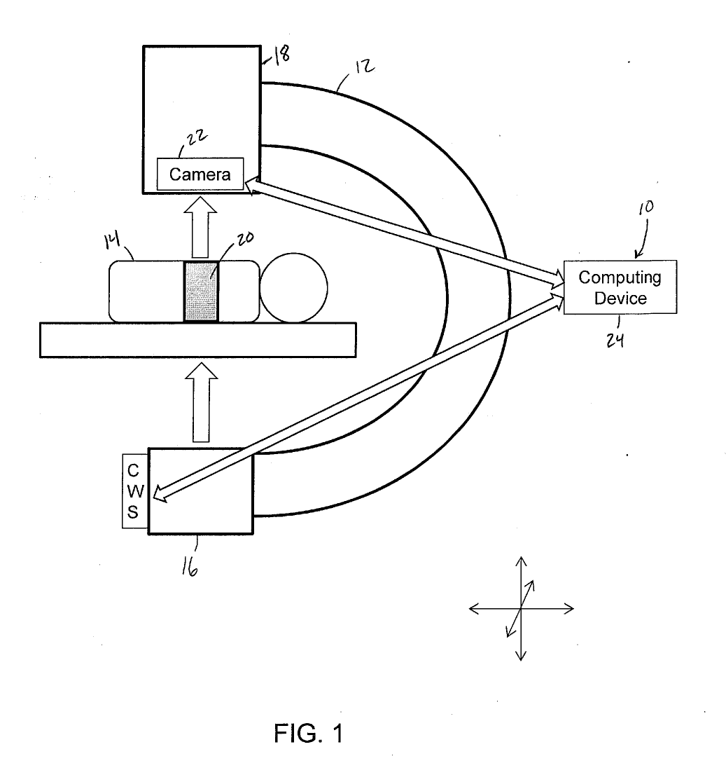

With reference to the drawings, and FIG. 1 in particular, in accordance with an aspect of the present, an intraoperative tracking and aligning apparatus 10 for a medical imaging device 12 is configured to be used in an operating room during patient 14 surgeries including orthopedic, vascular and neurosurgeries. By way of example and without limitation thereto, medical imaging device 12 may be a C-arm X-ray machine including an x-ray source 16 and image intensifier 18. In practice, medical imaging device 12 is typically be used by radiology technicians who use the C-arm machine to take intraoperative X-ray images of a targeted location 20. While the high-resolution X-ray images taken by the C-arm can help guide the surgeon, the bulkiness of the “C-shaped” arm has caused inconveniences when moving the image intensifier to the desired spot.

Accordingly, intraoperative tracking and aligning apparatus 10 may help the radiology technician view the surgical area 20 by providing a location tracking system for the C-arm position in the operating room and recording changes in angle and height of the arm. As will be discussed in greater detail below, a visualization device 22 alleviates the viewing problem caused by the bulkiness of the arm and gives the technician a better sense of the C-arm machine 12 position relative to the patient 14 and desired imaging location 20. In accordance with an aspect of the present invention, one non-limiting example of a visualization device 22 may include a 3D-camera, and more particularly a depth camera or RGB-D camera (red/green/blue-depth camera). In accordance with a further aspect of the present invention, the average time used to move the C-arm machine to the desired imaging location is shortened and fewer images are required in order to image the same location throughout the course of surgery. Therefore, the patient, surgeon, and technicians in the operating room all experience less radiation exposure.

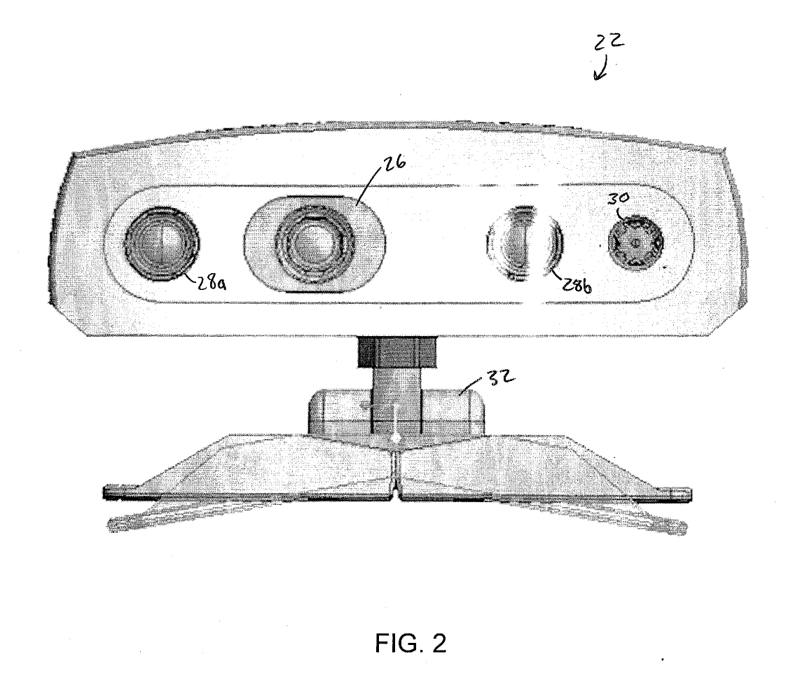

To that end, and with reference to FIGS. 1-3 in particular, in accordance with an aspect of the present, intraoperative tracking and aligning apparatus 10 includes a visualization device (camera) 22 mounted adjacent to image intensifier 18. Camera 22 may be mounted externally of the image intensifier housing or may be incorporated within the image intensifier housing. Camera 22 is in communication with a computing device 24, such as via a wired or wireless communication pathway. In accordance with an aspect of the present invention, an exemplary communication pathway may include Bluetooth or other WiFi communication. To that end, camera 22 and computing device 24 may include suitable transmitters and receivers configured to communication with one another within the operating room. As shown in FIG. 2, camera 22 may be an RGB-D camera including an infrared (IR) projector 26, stereo IR cameras 28a, 28b and color camera 30.

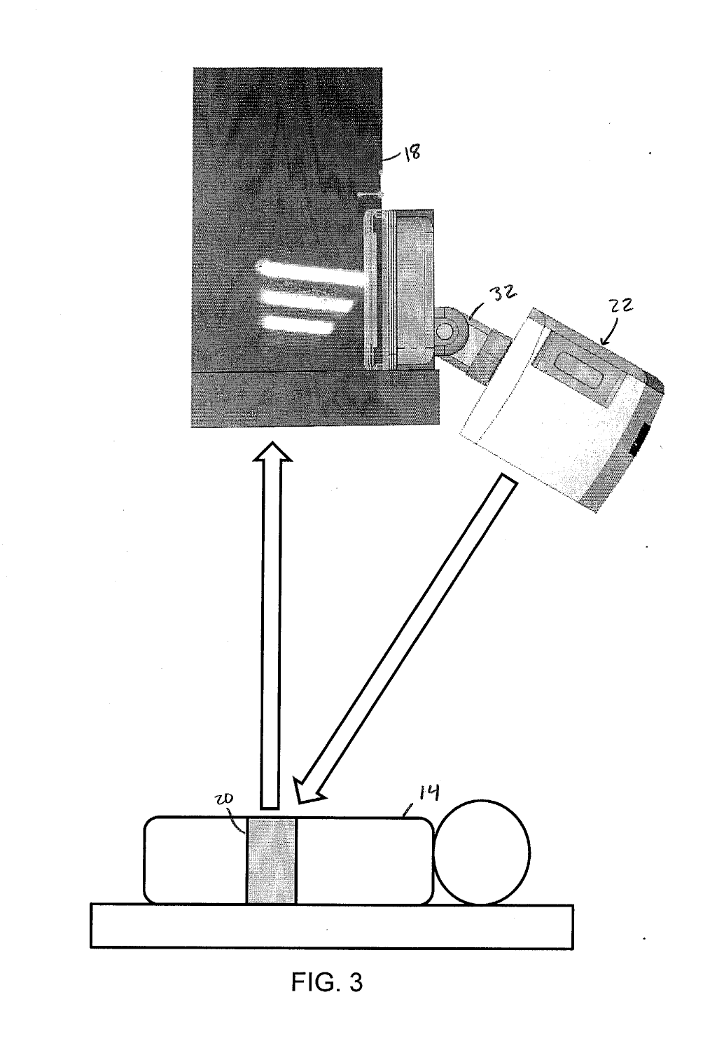

As shown in FIG. 3, camera 22 may be mounted onto image intensifier 18 through use of a swivel 32. Swivel 32 enables adjustment of the camera angle of camera 22. In accordance with an aspect of the present invention, the camera angle may be selected such that the field of view of camera 22 coincides substantially with surgical area 20 being irradiated by x-rays 34 emitted from x-ray source 16 and collected by image intensifier 18.

In accordance with a further aspect of the present invention, computing device 24 includes a processor and a memory and is programmed to include location tracking system (LTS) and surgical area visualization (SAV) software code. In this manner, the LTS, together with the SAV, is responsible to guide a user (e.g., x-ray technician) such that multiple x-ray images can be taken from the same spot even though medical imaging device 12 is moved In between the image captures. To that end, intraoperative tracking and aligning apparatus 10 is configured to capture one or more reference poses defined by RGB-D data received from 3D camera 22 from a specific angle and position, and visually guide the x-ray technician back to these reference poses when requested. Computing device 24 may be in communication with a user interface 36 to display the poses.

In accordance with another aspect of the present invention, the underlying tracking function in the LTS works by computing a pose difference between a first reference RGB-D image and a later-recorded RGB-D image. This pose difference is computed by first extracting a set of 3D features 37 from both the reference and later-recorded images. The software then searches for feature correspondences between the reference image and the later-recorded image by providing two feature sets. The feature sets are used to compute the 6D homogenous transformation between the sets by iteratively changing the pose while minimizing a cost function over the distance between the corresponding features 37. The software may additionally implement a hot starting mechanism to reduce the number of iterations and thereby increase the computation speed of the processor. The pose of the camera relative to the x-ray travel direction may be computed based on calibration information inserted by the x-ray technician and internal camera calibration parameters.

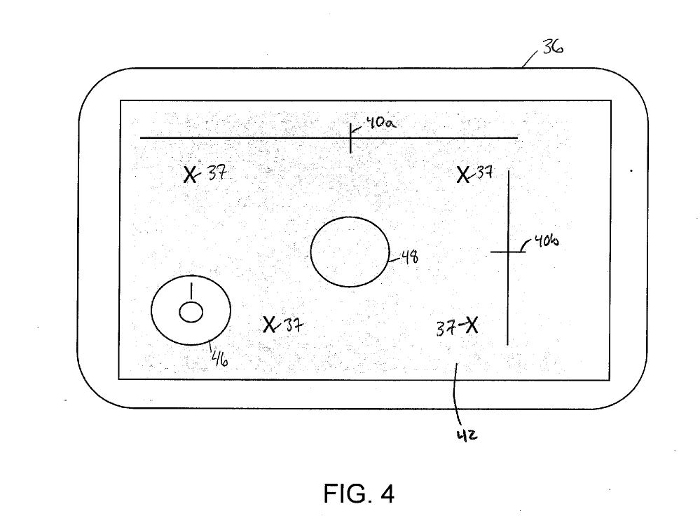

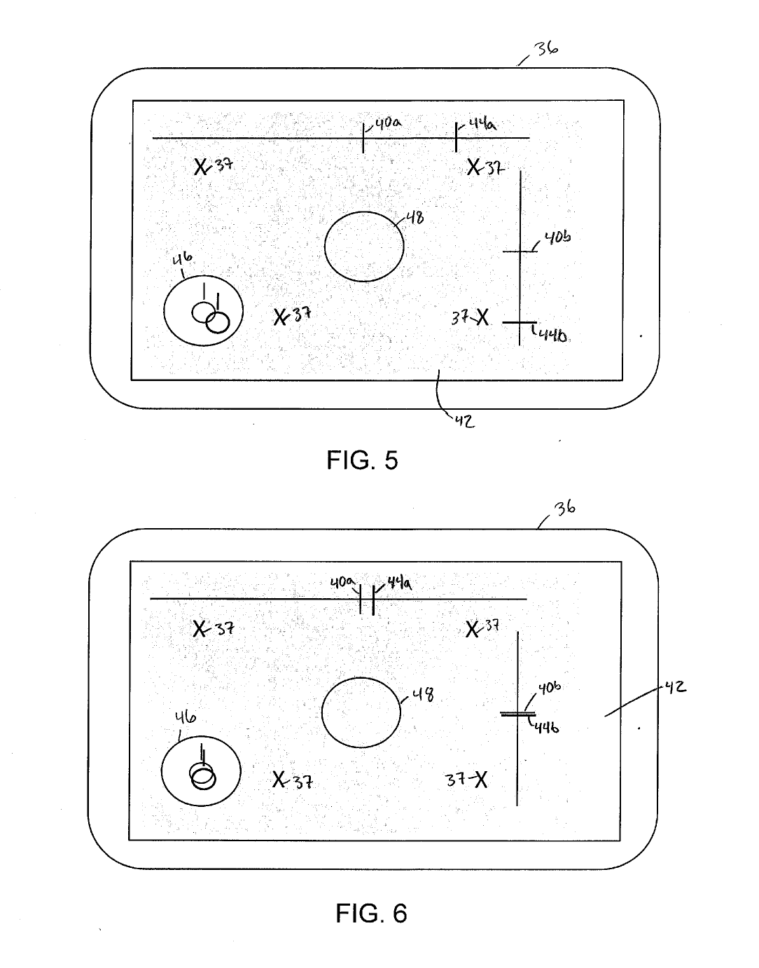

With reference to FIGS. 4-6, to facilitate realignment of medical imaging device 12 between successive images, the SAV may guide the x-ray technician to a reference pose in camera coordinate space rather than in reference pose coordinate space. To that end and as shown in FIG. 4, the reference pose is transformed into camera coordinate space 38 and sliders 40a, 40b are superimposed upon the reference image 42 displayed on the user interface 36. Subsequent image captures, such as when realigning medical imaging device 12, are analyzed such that second image sliders 44a, 44b are overlaid on reference image 42 and sliders 40a, 40b to show in which direction to move the camera with respect to the reference pose (see FIGS. 5 and 6). A compass ball 46 may also be used to guide the orientation of the system toward the reference. The x-ray impact area 48 may also indicated in the user interface, with the x-ray impact area depending on the distance from the camera to the object and the calibration of the camera relative to a travel direction of the x-rays emitted by the x-ray source.

In a further aspect of the present invention, a method for intraoperative tracking and aligning of a medical imaging device, the method comprises a) mounting a depth camera to the medical imaging device; b) providing a computing device including a processor and a memory, wherein the computing device is in communication with the depth camera; c) providing a user interface in communication with the computing device; d) acquiring first image data using the depth camera; e) communicating the first image data to the computing device to display a first image on the user interface; f) analyzing the first image data, via the processor, to identify a plurality of image features; g) superimposing a first alignment indicia over the first image; h) acquiring second image data using the depth camera; i) communicating the second image data to the computing device; j) analyzing the second image data, via the processor, to identify the plurality of image features; k) superimposing a second alignment indicia over the first image; and l) adjusting one or both of a location or orientation of the medical imaging device until the second alignment indicia coincides with the first alignment indicia.

In still another aspect of the present invention, with reference to FIG. 1, intraoperative tracking and aligning apparatus 10 may also include a collision warning system (CWS) 50. CWS 50 may be positioned on or within x-ray source 16 of medical imaging device 12. In practice, surgical procedures in the operating room include a drape placed over the operating table 52 while various tubes (e.g., IV tube, catheter, etc.) and leads (EKG, pulse oxygen, etc.) are attached to the patient. These tubes and leads may be suspended below the operating table 52 and may further be visually obscured by the drape. As a result, successive advancement and retreat of medical imaging device 12 may catch, and possibly pull, on one or more of these tubes and leads. To prevent unwanted engagement of medical imaging device 12 with anything located below operating table 52, CWS 50 may identify possible impacts/entanglements with x-ray source 16 and alert the x-ray technician. Repositioning of medical imaging device 12 may then be paused while any obstruction is removed or relocated. To that end, CWS 50 may include any suitable sensor or sensors, such as but not limited to a video camera, ultrasonic distance sensor or laser sensors. CWS 50 is in communication with computing device 24 such that user interface 36 emits an audio and/or video warning to the x-ray technician.

The foregoing description of the preferred embodiment of the invention has been presented for the purpose of illustration and description. It is not intended to be exhaustive nor is it intended to limit the invention to the precise form disclosed. It will be apparent to those skilled in the art that the disclosed embodiments may be modified in light of the above teachings. The embodiments described are chosen to provide an illustration of principles of the invention and its practical application to enable thereby one of ordinary skill in the art to utilize the invention in various embodiments and with various modifications as are suited to the particular use contemplated. Therefore, the foregoing description is to be considered exemplary, rather than limiting, and the true scope of the invention is that described in the following claims.

What is claimed is:

1. A method for intraoperative tracking and aligning of a medical imaging device, the method comprising;
a) mounting a depth camera to the medical imaging device;
b) providing a computing device including a processor and a memory, wherein the computing device is in communication with the depth camera;
c) providing a user interface in communication with the computing device;
d) acquiring first image data using the depth camera;
e) communicating the first image data to the computing device to display a first image on the user interface;
f) analyzing the first image data, via the processor, to identify a plurality of image features;
g) superimposing a first alignment indicia over the first image;
h) acquiring second image data using the depth camera;
i) communicating the second image data to the computing device;
j) analyzing the second image data, via the processor, to identify the plurality of image features;
k) superimposing a second alignment indicia over the first image;
l) adjusting one or both of a location or orientation of the medical imaging device until the second alignment indicia coincides with the first alignment indicia.

2. The method of claim 1 wherein steps f) and j) further include transforming the plurality of image features within the first image data and the second image data, respectively, into camera coordinate space.

3. The method of claim 2 wherein the first and second alignment indicia include first and second slider bars and a compass ball.

4. The method of claim 1 wherein the depth camera is configured to mount adjacent to an image intensifier of a C-arm x-ray machine, the image intensifier being located opposite an x-ray source.

5. The method of claim 4 further comprising adjusting a camera angle of the depth camera relative to a travel direction of x-rays emitted by the x-ray source.

6. The method of claim 5 wherein the depth camera is mounted onto a swivel configured to allow adjustment of the camera angle.

7. The method of claim 1 wherein the computing device includes the user interface.

8. An intraoperative tracking and aligning apparatus for a medical imaging device comprising;
a) a depth camera configured to mount to the medical imaging device;
b) a computing device including a processor and a memory, wherein the computing device is in communication with the depth camera;
c) a user interface in communication with the computing device;
wherein the depth camera acquires first image data and communicates the first image data to the computing device to display a first image on the user interface, wherein the processor analyzes the first image data to identify a plurality of image features and superimposes a first alignment indicia over the first image, and wherein the depth camera acquires second image data and communicates the second image data to the computing device, wherein the processor analyzes the second image data to identify the plurality of image features and superimposes a second alignment indicia over the first image, and
wherein one or both of a location or orientation of the medical imaging device is adjusted until the second alignment indicia coincides with the first alignment indicia.

9. The apparatus of claim 8 further comprising:
d) a collision warning system configured to mount to the medical imaging device, wherein the collision warning system is in communication with the computing device and is configured to trigger a warning signal when a possible collision is sensed.

10. The apparatus of claim 9 wherein the collision warning system comprises one or more of a video camera, an ultrasonic distance sensor or a laser sensor.
